(19) United States
(12) Patent Application Publication
(10) Pub. No.: US 2025/0169772 A1
(43) Pub. Date: May 29, 2025

(54) A HEADGEAR

(71) Applicant: TVS MOTOR COMPANY LIMITED, Chennai (IN)

(72) Inventors: Chaitanya Rajendra Zanpure, Chennai (IN); Abhishek Verma, Chennai (IN); Mummidivarapu Vineel Chandra, Chennai (IN); Datta Rajaram Sagare, Chennai (IN); K Venkata Manga Raju, Chennai (IN)

(73) Assignee: TVS MOTOR COMPANY LIMITED, Chennai (IN)

(21) Appl. No.: 18/851,518

(22) PCT Filed: Mar. 6, 2023
(86) PCT No.: PCT/IN2023/050206

§ 371 Date: Sep. 26, 2024

(30) Foreign Application Priority Data
Mar. 26, 2022 (IN) 202241017520

(51) Int. Cl. A61B 5/00; A42B 3/04; A61B 5/026; A61B 5/18; A61B 5/256; A61B 5/291; G06V 20/50; G06V 40/20; H04N 23/57

(52) U.S. Cl. CPC A61B 5/746; A42B 3/042; A61B 5/0261; A61B 5/18; A61B 5/256; A61B 5/291; A61B 5/6803; A61B 5/7405; A61B 5/7455; G06V 20/50; G06V 40/20; H04N 23/57

(57) ABSTRACT

A headgear includes a shell including a shell exterior and a shell interior; a visor connected to the shell; an electroencephalogram (EEG) sensor in the shell interior that generates a first signal indicative of state of a brain of a rider; a photoplethysmogram (PPG) sensor in the shell interior that generates a second signal indicative of blood flow rate in the rider's brain; an image sensor disposed in the shell interior that captures an image of the rider and generate image data; and a processor that: receives the first signal from the EEG sensor; receives the second signal from the PPG sensor; receives the image data from the image sensor; determines an attention score of the rider based on the first signal, the second signal, and the image data; and generates an alert signal when the attention score is below a pre-defined threshold value.

### FIELD OF THE INVENTION

The present invention relates to a headgear and a method for detecting drowsiness of a rider of a vehicle.

### BACKGROUND OF THE INVENTION

One of the major causes for vehicle accidents is drowsiness and fatigue of a rider of a vehicle. While leading car companies have come up with some or the other systems to address such issues, two-wheeled vehicles do not have such systems. The major challenge is to measure a real time fatigue or drowsiness level and provide alert to the rider.

Typically, for two-wheeled vehicle riders, measurement of fatigue level comes with challenges as the sitting arrangement is quite different as compared to other vehicles. One way to measure the fatigue level is to provide multiple sensors or gadgets on rider's body and process the signal obtained from each of the sensors or gadgets. However, such approach is practically not feasible because rider's body at all the times should be free from any hindrance. Moreover, rider's attention may get deviated due to multiple attachment of sensors or gadgets to the body of the rider.

Also, multiple EEG sensors are typically used to measure electrical activity of a brain. However, placing multiple sensors on the head of a rider while riding the motorcycle will necessarily cause discomfort which may deviate rider's attention.

Multiple number of sensors do not only cause discomfort to rider but also increases complexities of the system. With the increase in number of sensors, communication between a control unit and the sensors also becomes complex and costly.

Thus, there is a need in the art for a system which can measure and analyse bio-signals of a rider without causing discomfort to rider and which addresses at least the aforementioned problems.

### SUMMARY OF THE INVENTION

In one aspect, the present invention is directed towards a headgear having a shell exterior and a shell interior. The headgear has a visor connected to the shell; an electroencephalogram (EEG) sensor, a photoplethysmogram (PPG) sensor and an image sensor disposed in the shell interior; and a processor. The EEG sensor generates a first signal which is an indicative of state of a brain of a rider. The PPG sensor generates a second signal which is an indicative of blood flow rate in the rider's brain. The image sensor captures an image of the rider and generates image data. The processor receives the first signal from the EEG sensor, receives the second signal from the PPG sensor, and receive the image data from the image sensor. The processor determines an attention score of the rider based on the first signal, the second signal and the image data. The attention score is an indicative of a rider's drowsiness level. The processor further generates an alert signal if the attention score is below a pre-defined threshold value.

In an embodiment of the invention, the EEG sensor is disposed in vicinity of a prefrontal cortex region of the rider's head, the PPG sensor is disposed in vicinity of a middle portion of the rider's forehead and the image sensor is disposed adjacent to the visor.

In an embodiment of the invention, the PPG sensor measures the blood flow rate using low intensity infrared light and the EEG sensor measures a voltage difference between an active point and a reference point.

In an embodiment of the invention, the processor receives data which are indicative of vehicle riding parameters and image data which are indicative of behavioural parameters. The processor determines attention score of the rider based on the first signal, the second signal, the image data, and the vehicle riding parameters. In an embodiment, the vehicle riding parameters comprise frequent panic breaking, irregular steering, distance of a vehicle from a front vehicle, and a lean angle of the vehicle when the rider is riding the vehicle and is wearing the headgear. In an embodiment, the behavioural parameters comprise rider's head movements, duration between consecutive eye blinks, and yawning.

In another embodiment of the invention, the processor has a machine learning module which correlates the first signal, the second signal, and the image data and generate the attention score. In an embodiment, the machine learning module correlates the first signal, the second signal, the image data and the vehicle riding parameters and generates the attention score. In a further embodiment, the machine learning module determines rider's emotions. The machine learning module categorizes the rider's emotions as very weak, weak, strong, and very strong.

In a further embodiment of the invention, an Analog Front End (AFE) device is in communication with a Digital Signal Processor (DSP). The AFE device receives the first signal, the second signal, and the image data. The AFE device transmits amplified first signal, amplified second signal and amplified image data to the DSP. In an embodiment, the DSP which is communicatively coupled with the processor receives the amplified first signal, the amplified second signal, and the amplified image data. The DSP compares the amplified first signal, the amplified second signal, and the amplified image data with respective predetermined frequency range. The DSP transmits the amplified first signal, the amplified second signal, and the amplified image data within the predetermined frequency range to the processor.

In yet another embodiment of the invention, a communication module is provided which allows transmission of signals from the EEG sensor, the PPG sensor, and the image sensor to the processor. In an embodiment, the communication module allows transmission of signals from the EEG sensor, the PPG sensor, and the image sensor to the AFE device. In an embodiment, the communication module transmits and receives signals using Bluetooth protocol.

In another embodiment of the invention, an audio device is connected to the processor which receives the alert signal from the processor and generates a sound to alert the rider. In an embodiment, a haptic device is connected to the processor which receives the alert signal from the processor and generate a haptic feedback to alert the rider.

In another aspect, the present invention is directed towards a method for detecting drowsiness of a rider. The method comprises the step of generating a first signal which is an indicative of state of brain of a rider by an electroencephalogram (EEG) sensor which is disposed in a shell interior of a headgear. The method also comprises the step of generating a second signal which is an indicative of blood flow rate in the rider's brain by a photoplethysmogram (PPG) sensor which is disposed in the shell interior. The method further comprises the step of capturing an image of the rider by an image sensor which is disposed in the shell interior and generating image data. Thereafter, the first signal from the EEG sensor, the second signal from the PPG sensor and the image data from the image sensor is received by the processor. The method further comprises the step of determining an attention score of the rider based on the first signal, the second signal and the image data by the processor. The attention score is an indicative of the rider's drowsiness level. An alert signal is generated by the processor if the attention score is below a pre-defined threshold value.

In an embodiment of the invention, the method comprises the step of receiving data indicative of vehicle riding parameters by the processor and determining the attention score of the rider based on the first signal, the second signal, the image data, and the vehicle riding parameters by the processor.

In an embodiment of the invention, the method comprises the step of correlating the first signal, the second signal, and the image data and generating the attention score by a machine learning module of the processor. In another embodiment, the vehicle riding parameters together with the first signal, the second signal and the image data are correlated to generate the attention score by the machine learning module.

In an embodiment of the invention, the method comprises the step of determining rider emotions and categorizing the rider emotions as very weak, weak, strong, and very strong by the machine learning module.

In an embodiment of the invention, the method comprises the steps of receiving the first signal, the second signal, and the image data by an Analog Front End (AFE) device; transmitting an amplified first signal, an amplified second signal and an amplified image data to a Digital Signal Processor (DSP) by the AFE device.

In an embodiment of the invention, the method comprises the steps of receiving the amplified first signal, the amplified second signal, and the amplified image data by the DSP; comparing the amplified first signal, the amplified second signal, and the amplified image data with respective predetermined frequency range by the DSP; and transmitting the amplified first signal, the amplified second signal, and the amplified image data within a predetermined frequency range to the processor by the DSP.

In an embodiment of the invention, the method comprises the step of transmitting signals from the EEG sensor, the PPG sensor, and the image sensor to the processor by a communication module. In an embodiment, the signals are transmitted by the communication module to the AFE. In an embodiment, Bluetooth protocol is used by the communication module to transmit the signals.

In an embodiment of the invention, the method comprises the step of measuring the blood flow rate using low intensity infrared light by the PPG sensor.

In an embodiment of the invention, the method comprises the step of measuring a voltage difference between an active point and a reference point by the EEG sensor.

In an embodiment of the invention, the method comprises the steps of receiving the alert signal from the processor by an audio device; and generating a sound to alert the rider by the audio device. In an embodiment the method comprises the steps of receiving the alert signal from the processor by a haptic device; and generating a haptic feedback to alert the rider by the haptic device.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1 illustrates a schematic view of a headgear, in accordance with an embodiment of the invention.

FIG. 2 illustrates a block diagram of a headgear, in accordance with an embodiment of the invention.

FIG. 3 illustrates a method for detecting the drowsiness of a rider, in accordance with an embodiment of the invention.

FIG. 4 illustrates a D-vine copula distribution model, in accordance with an embodiment of the invention

### DETAILED DESCRIPTION OF THE INVENTION

Various features and embodiments of the present invention here will be discernible from the following further description thereof, set out hereunder.

The present invention generally relates to a headgear and a method 200 for detecting drowsiness of a rider.

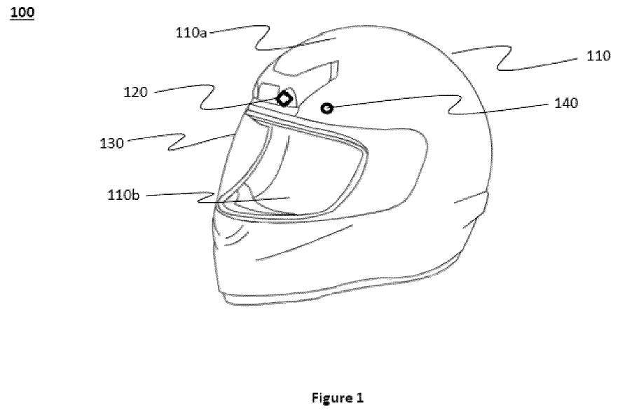

FIG. 1 illustrates a schematic view of a headgear 100, in accordance with an embodiment of the invention. The headgear 100 comprises a shell 110 which is configured to fit on a human head (not shown). The shell 110 has a shell exterior 110a and a shell interior 110b. Further, a visor 130 is connected to the shell 110.

The headgear 100 comprises an electroencephalogram (EEG) sensor 140 disposed in the shell interior 110b. In an embodiment, the EEG sensor 140 is disposed in vicinity of a prefrontal cortex region of a rider's head and attached in the shell interior 110b in such a way that it touches the prefrontal cortex region of the rider's head. The prefrontal cortex region of the brain plays a central role in cognitive control functions. The presence of dopamine in the prefrontal cortex region is very crucial in almost all aspects of high order cognition. The presence of dopamine modulates cognitive control, thereby influencing attention, impulse inhibition, prospective memory, and cognitive flexibility. These features are very crucial in determining the state of rider's brain. Hence, the EEG sensor 140 disposed in vicinity of the prefrontal cortex region of the rider's head will give more relevant result than any other region of the brain. The EEG sensor 140 comprises of EEG electrodes which detect electrical potentials in specific scalp region of the head. In this regard, the EEG sensor 140 is configured to measure a voltage difference between an active point and a reference point and thereafter generate a first signal. Since the present invention uses one EEG sensor 140, the first signal is a single channel EEG data obtained from the EEG sensor 140. The first signal is an indicative of state of a brain of the rider who is riding a vehicle (not shown). Accordingly, the EEG sensor 140 enables non-invasive, unobtrusive EEG monitoring which is used to track the brain's electrical activity.

The headgear 100 further comprises a photoplethysmogram (PPG) sensor 120 disposed in the shell interior 110b. In an embodiment, the PPG sensor 120 is disposed in vicinity of a middle portion of the rider's forehead and attached in the shell interior 110b in such a way that it touches the middle portion of the rider's forehead. The PPG sensor is non-invasive and uses a light source and a photodetector at the surface of human skin to measure variation in blood circulation. As such, the PPG sensor 120 measures the blood flow rate using low intensity infrared light and generates a second signal. The second signal is indicative of blood flow rate in the rider's brain.

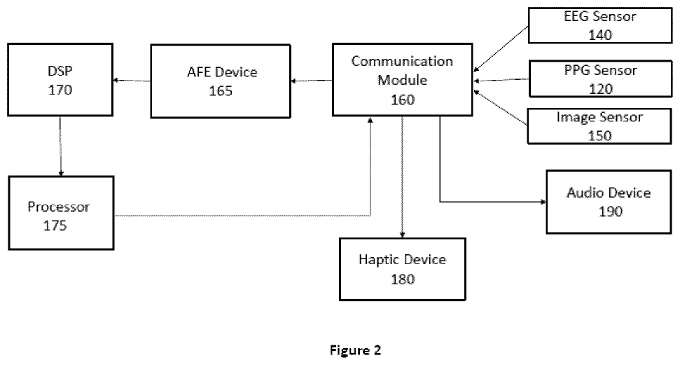

Referring to FIG. 2 in conjunction with FIG. 1, the headgear 100 comprises an image sensor 150 disposed in the shell interior 110b which captures an image of the rider and generate image data. In an embodiment, the image sensor 150 is disposed adjacent to the visor 130 facing the rider's face.

As shown in FIG. 2, the headgear 100 is provided with a communication module 160. The communication module 160 is in communication with the EEG sensor 140, the PPG sensor 120, and the image sensor 150 and configured to receive the first signal, the second signal, and the image data. The communication module 160 is further configured to transmit the first signal, the second signal and the image data to a processor 175. In this regard, an Analog Front End (AFE) device 165 and a Digital Signal Processor (DSP) 170 are provided.

The AFE device 165 is in communication with the communication module 160 and the DSP 170. The AFE device is configured to receive the first signal, the second signal, and the image data from the communication module 160, amplify the signals and the data, and transmit an amplified first signal, an amplified second signal and an amplified image data to DSP 170. In an embodiment, the communication module 160 transmits and receives signals using Bluetooth protocol.

The DSP 170 is communicatively coupled with the processor 175 and configured to receive the amplified first signal, the amplified second signal, and the amplified image data from the AFE device 165. The DSP 170 is further configured to compare the amplified first signal, the amplified second signal, and the amplified image data with a respective predetermined frequency range, and transmit the amplified first signal, the amplified second signal, and the amplified image data within the predetermined frequency range to the processor 175. In this regard, power spectral density of the first signal and heart rate variability from the second signal are by DSP 170.

The processor 175 is configured to receive the first signal from the EEG sensor 140, the second signal from the PPG sensor 120, and the image data from the image sensor 150. In an embodiment, as shown in FIG. 2, the processor 175 is communicatively coupled with the DSP 170 and receives the amplified first signal, the amplified second signal, and the amplified image data from the processor 175.

The processor 175 analyses the image data or the amplified image data and obtain behavioural parameters of the rider. In an embodiment, the behavioural parameters comprise rider's head movements, duration between consecutive eye blinks, yawning, and the like. In order to obtain the behavioural parameters, video feed in the form of image data is correlated. For example, if duration between consecutive eye blinks is higher than a predetermined time, then it indicates that the rider may be feeling sleepy. In another example, if the rider is yawning that means rider may be feeling sleepy. In another example, if eyes of the rider are closed and there is a sudden jerk in the head movement then it indicates that the rider was asleep.

In an embodiment, the processor 175 is configured to receive data which are indicative of vehicle riding parameters. These vehicle riding parameters include but are not limited to frequent panic breaking, irregular steering, distance of a vehicle from a front vehicle, and a lean angle of the vehicle when the rider is riding the vehicle and is wearing the headgear 100.

In an embodiment, the processor 175 comprises a machine learning module which correlates the first signal, the second signal, and the image data and generate the attention score. In another embodiment, the machine learning module correlates the first signal, the second signal, the image data together with the vehicle riding parameters and generate an attention score for the rider. The attention score indicative of the rider's drowsiness level or a fatigue level. In an embodiment, the machine learning module determines rider emotions. Further, the machine learning module categorizes the rider emotions such as, very weak, weak, strong, and very strong.

In order to perform the correlations, a multivariate vine copula is calculated. In an embodiment, the multivariate vine copula is a regular vine. The regular vine comprises a plurality of nodes connected by a plurality of edges. The plurality of nodes corresponds to plurality of vectors. The plurality of vectors is created for each of the EEG signal, the PPG signal, the behavioural parameters and the vehicle riding parameters. The plurality of edges represents a degree of dependence between each of the plurality of nodes. Further, the attention score is computed based on the multivariate vine copula. In an embodiment, the multivariate vine copula may be formed to estimate the attention score of the rider at a later point of time and if the attention score is less than a pre-defined value then the rider is alerted at a time instant before the attention score falls below the pre-defined value.

A “copula refers to a multivariate probability distribution of a multivariate dataset (e.g. the dataset of the values received from the EEG sensor or the PPG sensor), which may be used to decouple dependencies among various dimensions of the multivariate dataset. In an embodiment, the copula may be represented as a function of constituent univariate marginal distributions of the various dimensions in the multivariate dataset. In an embodiment, the univariate marginal distributions may be uniformly distributed. The overall copula density may be computed based on Copular Pair Densities and Marginal Densities.

$$\begin{matrix}Copula Density = F ( Copular Pair Densities \times Marginal Densities ) \\ Marginal Densities = F 4 ( V 4 ) \cdot F 3 ( V 3 ) \cdot F 2 ( V 2 ) \cdot F 1 ( V 1 )\end{matrix}$$

In an embodiment, an m-dimensional copula may be represented as a multivariate distribution function

C: [0,1]^{m}→[0,1]

The following equation represents a relationship between a joint distribution function F and univariate marginal distributions F₁(X₁), F₂(X₂), . . . Fₘ(Xₘ) of an m-dimensional multivariate dataset using an m-dimensional Copula function C:

$$\to F ( {X}_{1} , {X}_{2} , … , {X}_{m} ) = C ( {F}_{1} ( {X}_{1} ) , {F}_{2} ( {X}_{2} ) , … {F}_{m} ( {X}_{m} ) ) \qquad Equation 1$$

where,
Xi: a random variable for the i^{th} dimension of the m-dimensional multivariate dataset (e.g., a measure of a EEG Signal or a PPG signal in a multivariate dataset);
Fᵢ(Xᵢ): a univariate marginal distribution for the i^{th} dimension of the m-dimensional multivariate dataset, where Uᵢ≤Fᵢ(Xᵢ), Uᵢ: a cumulative distribution of Xᵢ; F( ) a joint distribution function of the m-dimensional multivariate dataset; and C( ): an m-dimensional copula function.

A “joint density function” refers to a joint probability distribution of a multivariate dataset. In an embodiment, the joint density function may represent a probability of assigning values to various dimensions of the multivariate dataset within a respective range associated with each dimension. In an embodiment, a joint density function ƒ of a m-dimensional multivariate dataset may be expressed in terms of an m-dimensional copula density function and univariate marginal density functions f₁, f₂, . . . fₘ as follows:

$$\to f ( {X}_{1} , {X}_{2} , … , {X}_{m} ) = {c}_{1} … m ( {F}_{1} ( {X}_{1} ) , {F}_{2} ( {X}_{2} ) , .. … {F}_{m} ( {X}_{m} ) ) \cdot {f}_{1} ( {X}_{1} ) \cdot {f}_{2} ( {X}_{2} ) … {f}_{m} ( {X}_{m} ) \qquad Equation 2$$

where,
f( ) a joint density function of the m-dimensional multivariate dataset;
fi(Xi): a marginal density function of Xi; and
c₁ . . . ₘ: an m-dimensional copula density function, where,

$$\to c 1 … m ( F 1 ( X 1 ) , F 2 ( X 2 ) , … F m ( X m ) ) = δ C δ F 1 δ F 2 … δ F m C ( F 1 ( X 1 ) , F 2 ( X 2 ) , … F m ( X m ) ) \qquad Equation 3$$

In an embodiment, the joint density function ƒ of the m-dimensional multivariate dataset may also be expressed in terms of conditional densities of the random variables as follows:

$$\to f ( {X}_{1} , {X}_{2} , … {X}_{m} ) = {f}_{m} ( {X}_{m} ) \cdot f ( {X}_{m - 1} / {X}_{m} ) … f ( {X}_{1} / {X}_{2} , … {X}_{m} ) \qquad Equation 4$$

where,
f(Xᵢ|Xₗ₊₁, . . . Xₗ₊ᵢ₋₁): a conditional density of the random variable Xₗ(for the I^{th} dimension),
where 1≤|≤m−1 and j=m−l.

By simplifying the equations 2 and 4, the joint density function f may be expressed in terms of univariate marginal density functions f₁, f₂, . . . fₘ and bivariate copula densities as follows:

$$\to f ( {X}_{1} , {X}_{2} , … {X}_{m} ) = {{\prod }_{k = 1}}^{m} {f}_{k} ( {X}_{k} ) {\prod }_{j = 1} {}^{m - 1} {\prod }_{I = 1} {( {}^{m - i} c )}_{I , I + j \vert I + 1 … I + j - 1} ( F ) {X}_{1} / {X}_{I + 1} , \cdot \cdot {X}_{I + j - 1} ) , F ) {X}_{I + j} / {X}_{i + 1} , … {X}_{i + j - 1} ) ) \qquad Equation 5$$

where,
c_{l,l+j|l+1} . . . ₗ₊ⱼ₋₁: a density of a bivariate copula distribution C_{l,l+j|l+1} . . . ₗ₊ⱼ₋₁; and F(Xₗ|Xₗ₊₁, . . . Xᵢ₊ⱼ₋₁): a conditional cumulative distribution of the random variable Xᵢ.

A “bivariate copula distribution” refers to a copula distribution that may model a dependency between a pair of dimensions of a multivariate dataset. For example, dependency between EEG signal data, PPG data, no of eye blinks and various other parameters that are part of the multivariate dataset are checked for interdependency by modelling the pair of dimensions.

Examples of the bivariate copula distribution may include, but are not limited to, a T-student copula distribution, a Clayton copula distribution, a Gumbel copula distribution, or a Gaussian copula distribution. In an embodiment, the bivariate copula distribution may be a part of a D-vine copula distribution.

A “d-vine copula” refers to a hierarchal collection of bivariate copula distributions. In an embodiment, the d-vine copula may be represented graphically by a set of hierarchal trees, each of which may include a set of nodes arranged sequentially and connected by a set of edges. Further, each edge, connecting a pair of nodes in a hierarchal tree, may represent a bivariate copula distribution. In an embodiment, for “m” random variables, the d-vine copula may correspond to a hierarchal structure including m−1 hierarchal trees representing a total of m (m−1)/2 bivariate copula distributions.

For example, a d-vine copula may be used to represent the bivariate copula distributions of the equation 5. In such a scenario, the variable j in the equation 5 may identify a hierarchal tree of the d-vine copula and the variable l in the equation 5 may identify an edge within that hierarchal tree, for representing each bivariate copula distribution of the equation 5 through the d-vine copula. In an embodiment, the d-vine copula may model a dependency between each pair of dimensions in a multivariate dataset. In an embodiment, the constituent bivariate copula distributions within the d-vine copula model may belong to different families of copula functions. Examples of the various families of copula functions include, but are not limited to, a T-student copula distribution, a Clayton copula distribution, a Gumbel copula distribution, or a Gaussian copula distribution.

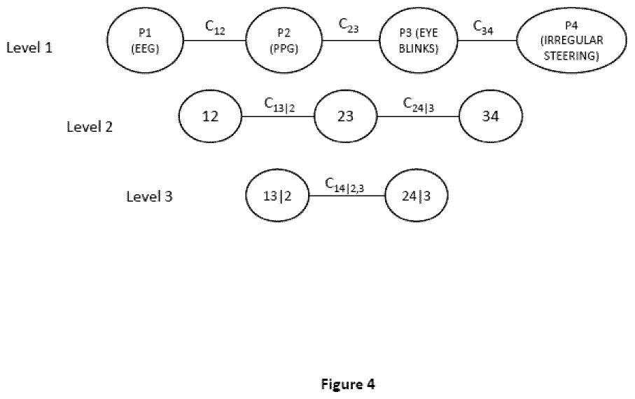

As illustrated in FIG. 4, an example of a D-vine copula distribution model, in accordance with at least one embodiment. In an embodiment, the D-vine copula corresponds to a scenario in which the multivariate data includes four parameters, for example, P1 (EEG data), P2 (PPG data), P3 (no of eye blinks), and P4 (irregular steering). In an actual implementation scenario, each of the parameters mentioned as part of the vehicle riding parameters may be used to compute the alertness of the rider.

The D-vine copula in the FIG. 4, may include three hierarchal trees (i.e., m−1 hierarchal tree, where m: number of parameters). A hierarchal tree at a particular level of the D-vine copula may include a sequence of connected nodes. In an embodiment, the tree at the first level of the D-vine copula may represent the various parameters in the multivariate data. Thus, the number of nodes at the first level may be same as the number of the parameters that need to be correlated. Further, the tree at the first level may represent bivariate copula distributions between pairs of parameters. In an embodiment, the tree at each subsequent level may represent bivariate copula distributions of the preceding level and conditional bivariate copula distributions determined based on such bivariate copula distributions of the preceding level.

For instance, the tree at the level 1 of the D-vine copula includes four nodes representing the four physiological parameters P1, P2, P3, and P4 respectively. The nodes are sequentially connected by edges, where each edge represents a bivariate copula distribution between the respective physiological parameters. For example, the edges connect each of the nodes. The edges may represent the bivariate copula and essentially represent correlation or dependency amongst the nodes. Further, the tree at the level 2 of the D-vine copula includes three nodes. Each of the three nodes may represent a corresponding bivariate copula represented at the previous level. As illustrated before in level 1, the edges connect each of the nodes and the edges may represent the bivariate copula and essentially represent correlation or dependency amongst the nodes.

Further, the nodes at the level 2 of the D-vine copula, may be sequentially connected by edges. Each edge between a pair of nodes at the level 2 may represent a conditional bivariate copula, which may be determined based on the pair of bivariate copulas, represented by the pair of nodes. The edges in level 2 may represent the conditional bivariate copulas.

In addition, the tree at the level 3 of the D-vine copula includes two nodes. The first node in level 3 may correspond to the first edge of the previous level, i.e., the level 2. Further, the second node in level 3 may correspond to the second edge of the level 2. Hence, the first node may denote the conditional bivariate copula C13|2, which is represented by the corresponding edge. Similarly, the second node may denote the conditional bivariate copula C24|3, which is represented by the corresponding edge. Further, the first nodes and second node may be connected by an edge. Such an edge may represent the conditional bivariate copula C14|3,2, which may be determined based on the conditional bivariate copulas C13|2 and C24|3.

A person skilled in the art will understand that though the D-vine copula has been illustrated for an example scenario of four parameters, the D-vine copula may be similarly extended for any number of parameters. In an embodiment, the number of levels of the D-vine copula may be given by m−1 and the number of bivariate copulas represented by the D-vine copula may be given by m(m−1)/2, where m: number of parameters.

After determining the attention score, the processor generates an alert signal if the attention score is below a pre-defined threshold value. In this regard, as shown in FIG. 2, an audio device is provided to receive the alert signal from the processor 175 and generate a sound to alert the rider. Alternatively, a haptic device 180 is provided to receive the alert signal from the processor 175 and generate a haptic feedback to alert the rider. In an embodiment, the haptic feedback may be provided on a handlebar grip.

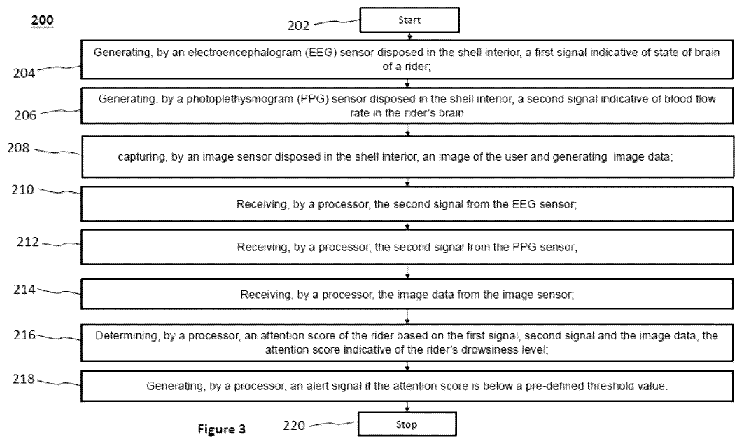

In another aspect, the present invention relates to a method 200 for detecting drowsiness of the rider, as referenced above. FIG. 3 illustrates, the method steps involved in the method 200. At step 204, the first signal is generated by the EEG sensor 140 which is disposed in the shell interior 110b of the headgear 100. In an embodiment, the method 200 comprises the step of measuring a voltage difference between an active point and a reference point by the EEG sensor 140. The first signal is the indicative of state of brain of the rider.

At step 206, the second signal is generated by the PPG sensor 120. The PPG sensor 120 is disposed in the shell interior 110b of the headgear 100. In an embodiment, the method 200 comprises the step of measuring the blood flow rate using low intensity infrared light by the PPG sensor 120. The second signal is the indicative of blood flow rate in the rider's brain.

At step 208, the image of the rider is captured, and the image data is generated by the image sensor 150. The image sensor 150 is disposed in the shell interior 110b.

In an embodiment, the method 200 comprise the steps of transmitting signals from the EEG sensor 140, the PPG sensor 120, and the image sensor 150 to the processor 175 by the communication module 160. Alternatively, the first signal, the second signal and the image data are received by the AFE device 165. The AFE device 165 amplifies the signals and transmit amplified first signal, amplified second signal and amplified image data to the DSP 170. The DSP compares the amplified first signal, the amplified second signal, and the amplified image data within a predetermined frequency range and transmits the amplified first signal, the amplified second signal, and the amplified image data within a predetermined frequency range to the processor 175.

At step 210, the processor 175 receives the first signal from the EEG sensor 140. At step 212, the processor 175 receives the second signal from the PPG sensor 120. At step 214, the processor 175 receives the image data from the image sensor 150. At this stage, the processor 175 correlates the first signal, the second signal, and the image data by the machine learning module of the processor 175.

At step 216, an attention score of the rider is calculated based on the first signal, the second signal and the image data by the machine learning module of the processor 175. The attention score is an indicative of the rider's drowsiness level. In an embodiment, the step 216 of method 200 comprise receiving data by the processor 175 which are indicative of vehicle riding parameters. These vehicle riding parameters include but are not limited to frequent panic breaking, irregular steering, distance of a vehicle from a front vehicle, and the lean angle of the vehicle. The rider is riding the vehicle and is wearing the headgear 100.

In another embodiment, the attention score of the rider can also be calculated based on the first signal, the second signal, the image data, and the vehicle riding parameters by the machine learning module of the processor 175. At step 218, the alert signal is generated by the processor 175 if the attention score is below the pre-defined threshold value.

In a further embodiment, the machine learning module determines rider's emotions. The machine learning module categorizes the rider's emotions as very weak, weak, strong, and very strong.

Advantageously, the present invention provides better safety to the rider while riding the vehicle. The present invention provides the headgear 100 which monitors the alertness of the rider by measuring the blood flow rate using PPG sensor 120 and the rider's brain activity using EEG sensor 140 and various other behavioural parameters using the image sensor 150. The invention reduces the risk of automobile accidents by alerting the rider whenever the attention score is below the pre-defined threshold value.

Further, the present invention ensures a better cost management of the product by reducing the number of sensors. Hence, making the headgear 100 more economically feasible. Moreover, it just not only reduces the cost but also reduces the complexity of the headgear 100 by using the single channel EEG data.

The claimed steps as discussed herein are not routine, conventional, or well understood in the art, as the claimed steps enable the following solutions to the existing problems in conventional technologies. In the headgear 100, the EEG sensor 140 is disposed in vicinity of the prefrontal cortex region of the rider's head and attached in the shell interior 110b in such a way that it touches the prefrontal cortex region of the rider's head. Due to such disposition of the EEG sensor 140, it generates the first signal which is indicative of state of brain of the rider. Moreover, due to single EEG sensor 140, a single channel EEG data obtained from the EEG sensor 140. The PPG sensor 120 generates the second signal which is indicative of blood flow rate in the rider's brain. The PPG sensor 120 is disposed in vicinity of the middle portion of the rider's forehead to obtain the blood flow rate accurately. The image sensor 150 captures the image of the rider and generates image data. The processor 175 receives the first signal from the EEG sensor 140, receives the second signal from the PPG sensor 120, and receive the image data from the image sensor 150. The processor 175 determines an attention score of the rider based on the first signal, the second signal and the image data. The attention score is the indicative of the rider's drowsiness level. The processor 175 further generates the alert signal if the attention score is below a pre-defined threshold value. Thus, the usage of single channel EEG reduces the structural complexity of the headgear 100. In the present invention, the minimum number of sensors are used in order to ensure that the rider's attention does not get deviated. The necessary comfort of the rider will be maintained while riding. By reducing the usage of multiple number of sensors, it not only causes comfort to rider but also reduces the complexities of the system. The communication between the control unit and the sensors also becomes less complex and less costly due to the presence of minimum number of sensors. Furthermore, processing time for processing the data received by the processor 175 is greatly reduced.

While the present invention has been described with respect to certain embodiments, it will be apparent to those skilled in the art that various changes and modification may be made without departing from the scope of the invention as defined in the following claims.

1.-33. (canceled)

34. A headgear comprising:
a shell comprising a shell exterior and a shell interior;
a visor connected to the shell;
an electroencephalogram (EEG) sensor disposed in the shell interior and configured to generate a first signal indicative of state of a brain of a rider;
a photoplethysmogram (PPG) sensor disposed in the shell interior and configured to generate a second signal indicative of blood flow rate in the rider's brain;
an image sensor disposed in the shell interior and configured to capture an image of the rider and generate image data; and
a processor configured to:
receive the first signal from the EEG sensor; receive the second signal from the PPG sensor;
receive the image data from the image sensor;
determine an attention score of the rider based on the first signal, the second signal, and the image data, the attention score indicative of a drowsiness level of the rider; and
generate an alert signal when the attention score is below a pre-defined threshold value.

35. The headgear according to claim 34, wherein the processor is configured to:
receive data indicative of vehicle riding parameters;
receive image data indicative of behavioral parameters; and
determine attention score of the rider based on the first signal, the second signal, the image data, and the vehicle riding parameters.

36. The headgear according to claim 34, wherein the processor comprises a machine learning module configured to:
correlate the first signal, the second signal, and the image data; and
generate the attention score.

37. The headgear according to claim 36, wherein the machine learning module is configured to:
correlate the first signal, the second signal, the image data and vehicle riding parameters; and
generate the attention score.

38. The headgear according to claim 36, wherein the machine learning module is configured to:
determine rider's emotions; and
categorize the rider's emotions as very weak, weak, strong, and very strong.

39. The headgear according to claim 35, wherein the vehicle riding parameters comprise frequent panic breaking, irregular steering, distance of a vehicle from a front vehicle, and lean angle of the vehicle, wherein the rider is riding the vehicle and is wearing the headgear.

40. The headgear according to claim 34, further comprising:
an Analog Front End (AFE) device; and
a Digital Signal Processor (DSP) in communication with the AFE, the AFE device configured to:
receive the first signal, the second signal, and the image data; and
transmit amplified first signal, amplified second signal and amplified image data to the DSP.

41. The headgear according to claim 40, wherein the DSP is communicatively coupled with the processor and is configured to:
receive the amplified first signal, the amplified second signal, and the amplified image data;
compare the amplified first signal, the amplified second signal, and the amplified image data with respective predetermined frequency range; and
transmit the amplified first signal, the amplified second signal, and the amplified image data within the predetermined frequency range to the processor.

42. The headgear according to claim 40, further comprising a communication module configured to allow transmission of signals from the EEG sensor, the PPG sensor, and the image sensor to the processor.

43. The headgear according to claim 42, wherein the communication module is configured to allow transmission of signals from the EEG sensor, the PPG sensor, and the image sensor to the AFE device.

44. The headgear according to claim 42, wherein the communication module is configured to transmit and receive signals using Bluetooth protocol.

45. The headgear according to claim 35, wherein the behavioral parameters include rider's head movements, duration between consecutive eye blinks, and yawning.

46. The headgear according to claim 34, wherein the EEG sensor is disposed in vicinity of a prefrontal cortex region of a head of the rider.

47. The headgear according to claim 34, wherein the PPG sensor is disposed in vicinity of a middle portion of a forehead of the rider.

48. The headgear according to claim 34, wherein the image sensor is disposed adjacent to the visor.

49. The headgear according to claim 34, wherein the PPG sensor is configured to measure the blood flow rate using low intensity infrared light.

50. The headgear according to claim 34, wherein the EEG sensor is configured to measure a voltage difference between an active point and a reference point.

51. The headgear according to claim 34, further comprising an audio device connected to the processor and is configured to:
receive the alert signal from the processor; and
generate a sound to alert the rider.

52. The headgear according to claim 34, further comprising a haptic device connected to the processor and is configured to:
receive the alert signal from the processor; and
generate a haptic feedback to alert the rider.

53. A method for detecting drowsiness of a rider, the method comprising:
generating, by an electroencephalogram (EEG) sensor disposed in a shell interior of a headgear, a first signal indicative of state of brain of a rider;
generating, by a photoplethysmogram (PPG) sensor disposed in the shell interior, a second signal indicative of blood flow rate in the rider's brain;
capturing, by an image sensor disposed in the shell interior, an image of the rider;
generating image data;
receiving, by a processor, the first signal from the EEG sensor;
receiving, by the processor, the second signal from the PPG sensor;
receiving, by the processor, the image data from the image sensor;
determining, by the processor, an attention score of the rider based on the first signal, the second signal and the image data, the attention score indicative of a drowsiness level of the rider; and
generating, by the processor, an alert signal when the attention score is below a pre-defined threshold value.

54. The method according to claim 53, further comprising:
receiving, by the processor, data indicative of vehicle riding parameters; and
determining, by the processor, the attention score of the rider based on the first signal, the second signal, the image data, and the vehicle riding parameters.

55. The method according to claim 53, further comprising:
correlating, by a machine learning module of the processor, the first signal, the second signal, and the image data; and
generate the attention score.

56. The method according to claim 55, further comprising:
correlating, by the machine learning module, the first signal, the second signal, the image data and vehicle riding parameters; and
generating, by the machine learning module, the attention score.

57. The method according to claim 55, further comprising:
determining, by the machine learning module, rider emotions; and
categorizing, by the machine learning module, the rider emotions as very weak, weak, strong, and very strong.

58. The method according to claim 53, further comprising:
receiving, by an Analog Front End (AFE) device, the first signal, the second signal, and the image data; and
transmitting, by the Analog Front End (AFE) device, amplified first signal, amplified second signal and amplified image data to a Digital Signal Processor (DSP).

59. The method according to claim 58, further comprising:
receiving, by the DSP, the amplified first signal, the amplified second signal, and the amplified image data;
comparing, by the DSP, the amplified first signal, the amplified second signal, and the amplified image data with respective predetermined frequency range; and
transmitting, by the DSP, the amplified first signal, the amplified second signal, and the amplified image data within a predetermined frequency range to the processor.

60. The method according to claim 53, further comprising transmitting, by a communication module, signals from the EEG sensor, the PPG sensor, and the image sensor to the processor.

61. The method according to claim 58, further comprising transmitting, by a communication module, signals from the EEG sensor, the PPG sensor, and the image sensor to the AFE device.

62. The method according to claim 60, further comprising transmitting and receiving, by the communication module, signals using Bluetooth protocol.

63. The method according to claim 53, further comprising measuring, by the PPG sensor, the blood flow rate using low intensity infrared light.

64. The method according to claim 53, further comprising measuring, by the EEG sensor, a voltage difference between an active point and a reference point.

65. The method according to claim 53, further comprising:
receiving, by an audio device, the alert signal from the processor; and
generating, by an audio device, a sound to alert the rider.

66. The method according to claim 53, further comprising:
receiving, by a haptic device, the alert signal from the processor; and
generating, a haptic feedback to alert the rider.
